(19) United States
(12) Patent Application Publication
(10) Pub. No.: US 2025/0308655 A1
(43) Pub. Date: Oct. 2, 2025

(54) SYSTEM AND METHODS FOR PREVENTIVE CARE AND TESTING WITH INTEGRATION INTO SOCIAL MEDIA DATING APPLICATIONS

(71) Applicant: Michael Donathan, Scottsdale, AZ (US)

(72) Inventor: Michael Donathan, Scottsdale, AZ (US)

(21) Appl. No.: 19/096,656

(22) Filed: Mar. 31, 2025

Related U.S. Application Data
(60) Provisional application No. 63/573,350, filed on Apr. 2, 2024.

(51) Int. Cl. G16H 10/60; G06Q 50/00; G16H 50/20

(52) U.S. Cl. CPC G16H 10/60; G06Q 50/01; G16H 50/20

(57) ABSTRACT

A method and system for providing PCT (Preventive Care and Testing) services to customers can involve receiving user data from a plurality of users, including personal information and medical history, conducting PCT testing on the users for Sexually Transmitted Diseases (STDs), generating a certification indicating the STD-free status of the users based on the results of the PCT testing, and coordinating with dating services to incorporate the certification into user profiles, thereby indicating to potential partners the STD-free status of the users.

### CROSS REFERENCE TO PROVISIONAL APPLICATION

This patent application claims priority to U.S. Provisional Patent Application No. 63/573,350 entitled “SYSTEM AND METHODS FOR PREVENTIVE CARE AND TESTING WITH INTEGRATION INTO SOCIAL MEDIA DATING APPLICATIONS,” which was filed on Apr. 2, 2024, and is incorporated herein by reference in its entirety.

### TECHNICAL FIELD

Embodiments are generally related to dating applications. Embodiments are also generally related to preventive care and testing (PCT). Embodiments are more particularly directed to methods and systems involved in providing preventive care and testing privately in a direct to patient/consumer fashion and coordinating results with dating services to certify users as STD/STI-free.

### BACKGROUND

With the proliferation of dating apps and online platforms facilitating casual encounters, there has been a corresponding increase in the transmission of STDs (sexually transmitted diseases), also referred to as STIs (sexually transmitted diseases), among users. Despite the high prevalence of STDs/STIs, there is often underreporting due to social stigma, fear of judgment, and lack of awareness.

The sexual health and wellness market is growing rapidly, with an increasing emphasis on open communication and responsible behavior. Individuals are seeking ways to improve their sexual health awareness and make informed decisions.

What is needed are systems and methods for STD/STI testing that can also be embodied/integrated in an application whose target audience can include sexually active adults who value transparency and responsible sexual behavior.

### BRIEF SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

What is described in the embodiments described herein is a revolutionary system and methods that can be partially embodied in a mobile application, designed to empower individuals by providing a secure platform to manage and share their sexual health results, fostering transparency, responsible behavior, and credibility within intimate relationships. The inventive embodiments when technologically implemented and expressed as a service for users aim to reduce stigma, improve communication, and promote sexual health awareness, ultimately contributing to healthier and safer sexual experiences.

It is an aspect of the embodiments to enable users to access, store, and share their verified sexual health test results, including STD (sexually transmitted disease)/STI (sexually transmitted infection) tests, HIV tests, and other relevant health information. STI and STD will both be referred hereinafter as just “STD”.

It is another aspects of the embodiments to provide users with a profile badge denoting their STD-free status on dating apps, ensuring transparency and promoting safer interactions within the dating community.

According to an embodiment, a method is described for providing PCT (Preventive Care and Testing) services to customers that can include the steps of receiving user data from a plurality of users, including personal information and medical history, conducting PCT testing on the users for STDs, generating a certification indicating the STD-free status of the users based on the results of the PCT testing, and coordinating with dating services to incorporate the certification into user profiles, thereby indicating to potential partners the STD-free status of the users.

According to another embodiment, a system is described for coordinating PCT testing with dating services to certify the STD-free status of users. The system can include a database for storing user data, including personal information, medical history, and PCT test results, a PCT testing module configured to conduct STD testing on the users and generate test results, a certification generation module configured to create certifications indicating the STD-free status of the users based on the test results, and an interface module configured to communicate with dating services to integrate the certifications into user profiles.

According to yet another embodiment, a computer-readable storage medium for storing instructions can be provided that, when executed by a processor, can cause the processor to perform a method for certifying the STD-free status of users on dating services. The method can include steps of receiving user data from a plurality of users, including personal information and medical history, conducting PCT testing on the users for STDs, analyzing the results of the PCT testing to determine the STD-free status of the users, generating a certification indicating the STD-free status of the users, and transmitting the certification to dating services for incorporation into user profiles.

### BRIEF DESCRIPTION OF THE DRAWINGS

The accompanying figures, in which like reference numerals refer to identical or functionally similar elements throughout the separate views and which are incorporated in and form a part of the specification, further illustrate the present invention and, together with the detailed description of the invention, serve to explain the principles of the present invention.

FIG. 1 illustrates is a flow diagram, in accordance with an embodiment;

FIG. 2 illustrates a system diagram, in accordance with an embodiment; and

FIG. 3 illustrates a schematic diagram of an example operating environment in accordance with one or more implementations described herein.

### DETAILED DESCRIPTION

The particular values and configurations discussed in these non-limiting examples can be varied and are cited merely to illustrate one or more embodiments and are not intended to limit the scope thereof.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof (other than software per se). The following detailed description is, therefore, not intended to be interpreted in a limiting sense.

Throughout the specification and claims, terms may have nuanced meanings suggested or implied in context beyond an explicitly stated meaning. Likewise, phrases such as “in one embodiment” or “in an example embodiment” and variations thereof as utilized herein do not necessarily refer to the same embodiment and the phrase “in another embodiment” or “in another example embodiment” and variations thereof as utilized herein may or may not necessarily refer to a different embodiment. It is intended, for example, that claimed subject matter include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context. Furthermore, the term “at least one” as utilized herein can refer to “one or more”. For example, “at least one widget” may refer to “one or more widgets.”

The term “data” refers herein to physical signals that indicate or include information. An “image,” as a pattern of physical light or a collection of data representing the physical light, may include characters, words, and text as well as other features such as graphics.

The term “app” as utilized herein can relate to a software-implemented process executed on a computing device, such as a smartphone, tablet, or other electronic hardware, that performs a specific function or set of functions. The app can be configured to interact with device hardware, including input/output interfaces, memory storage, sensors, or network communication modules, to achieve a technical improvement over conventional methods. The app may also facilitate data processing, user interaction, or automated execution of tasks that enhance computational efficiency or functionality.

The app can be implemented on a computing device comprising a processor and a memory storing executable instructions that, when executed, cause the processor to perform a set of operations that improve the functionality of the device. An app can interact with specific hardware elements, such as sensors, GPS, or biometric scanners, processors and other devices and hardware components to perform the operations and features described herein, which were not previously achievable using conventional systems. Unlike conventional methods, the app leverages the disclosed operations and components to enhance processing speed, reduce computational load, or improve accuracy in preventive care and testing with integration into social media data applications. The app improves upon existing technology by, for example, reducing network latency, enhancing security protocols, improving resource allocation, and improving accuracy in preventive care and testing with integration into social media data applications. The claimed invention is not merely a generic execution of software but rather provides a novel data-processing technique that enhances the functionality of the computing device. The app implements a unique method, which operates in a manner distinct from conventional generic computing processes. Furthermore, the app can transform input data into a structured output that facilitates improvements in accuracy in preventive care and testing with integration into social media data applications, achieving a result that is not merely an abstract concept. The app can also integrate with external hardware or networked systems in some embodiments to effectuate real-world changes, such as controlling a physical device, optimizing resource distribution, or generating actionable insights, particularly related to improvements in accuracy in preventive care and testing with integration into social media data applications

Undergoing an STD test before each date from a dating site greatly enhances safety, reducing STD transmission risks and promotes open communication about sexual health. This practice not only protects individuals but also supports public health efforts, making the dating environment safer and more trustworthy. The present embodiments provide a system and methods which can be expressed in services that give people transparency and safety in dating through technology. Implementation of the embodiment describe herein can revolutionize the dating landscape by leveraging modern technology to empower individuals with comprehensive information and protection against STDs.

A sexual health credibility app has the potential to revolutionize the way individuals approach sexual health, communication, and responsibility. By providing a secure platform for sharing verified sexual health results, the app aims to foster transparency, trust, and informed decision-making within intimate relationships. As society becomes more open to discussing sexual health, this app can play a pivotal role in improving overall sexual well-being and reducing the stigma associated with sexual health discussions.

How the system works:

A user can first sign up for or create an account with an online service representing the sexual health credibility app.

Once signed up with the service, the user can request a test kit, and provide basic information (e.g., demographic, behavioral, data app registrations). Only information that is needed can be provided and it will remain private.

Service can be provided in a direct to consumer (or direct to patient) model of business for testing purposes. This alleviates the need for users to physically go to a clinic for occasional (e.g., monthly) testing. A Kit box can be received within 5-7 days after registering with the service and ordering the kit.

The kit can also contain instructions. Users can follow clear testing instruction in kit about how to conduct the STD test. After completing the tests, the user can return specimen to testing labs. Once results have been received, the service providers API technology can seamlessly work with dating apps the user is registered with to provide an official badge on the user's profile. The badge can be clearly marked on the front of the user's profile to let others using the same App know that the user has been verified as STD free. Openly sharing status helps reduce stigma, provides peace of mind, and encourages healthier sexual practices within the dating community.

Verified Results: The App can be partnered with certified medical testing facilities to ensure that users' sexual health results are authentic and accurate. The App can also be partnered with dating sites where users can upload results for credibility.

Privacy and Security: User data can be encrypted and stored securely, ensuring the utmost privacy. Users will have full control over who can access their information.

Relationship Enhancement: The app can promote open and honest communication between partners, fostering trust and responsible behavior.

Sexual Health Education: The App can offer educational content on sexual health, STIs, safe practices, and consent.

Premium Version (Paid Subscription): Subscribers can also enjoy advanced features such as priority verification, access to premium educational content, and anonymous peer support. Along with their uploaded STD results showing a Green or Blue Checkmark as certification. Other certification seals can also be devised and used. Premium membership can include automatic monthly shipments of testing kits so that users can be prepared should they have an unexpected encounter.

A service can also share online informative articles, infographics, and videos to engage users and encourage them to prioritize sexual health. The service can collaborate with health insurance companies, clinics, and healthcare providers to offer users additional value. By implementing robust data protection and privacy policies, the service can prioritize user confidentiality and trust, fostering a safe and secure environment for individuals to manage their sexual health with confidence. The service can ensure the utmost confidentiality of test results and personal information through rigorous measures. User data, including test results and personal details, are encrypted and stored securely, accessible only to authorized personnel.

Systems and methods described as part of the inventive embodiment can be implemented for a service that involves direct to consumer STD testing delivered to the patient's door. A monthly membership model can be implemented for users where the user has tests on hand and can test monthly to keep their “check mark” or “verified” status (e.g., a sexual health passport). HIPAA compliant software can be integrated with any dating site or be a stand-alone application or website that shows patients most recent results, discreet to the patient. The checkmark or verified status can show only when users have been tested and are current on their membership.

A system can provide a “click here to speak with provider” option if the user/member tests positive for something. This can direct them to telehealth providers for confidential treatment that can be connected to/sent to the affected users.

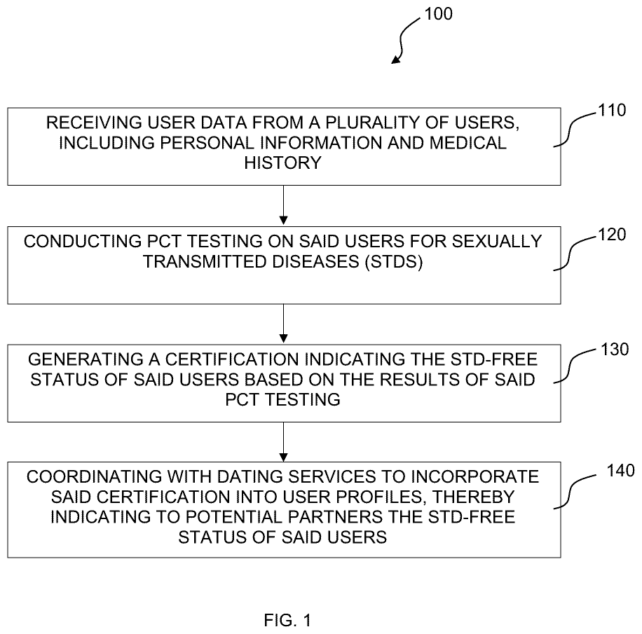

Referring to FIG. 1, illustrated is a flow diagram 100 of a method in accordance with an embodiment of the invention. Referring to Block 110, a testing system can receive user data from a plurality of users, information that can include personal information and medical history. Referring to Block 120, PCT testing can be conducted on the users for sexually transmitted diseases. Referring to Block 130, a certification can be generated by the system indicating the STD-free status of users based on the results of STD testing. Then, as shown in Block 140, the service/system can coordinate with dating services to incorporate the certification into user profiles, thereby indicating to potential partners the STD-free status of the users.

Testing methods and instruments can include those currently used my medical laboratories to include PCT (polymerase Chain Reaction), blood sampling, urine sampling, saliva testing, oral swabs, genital swabs, etc.

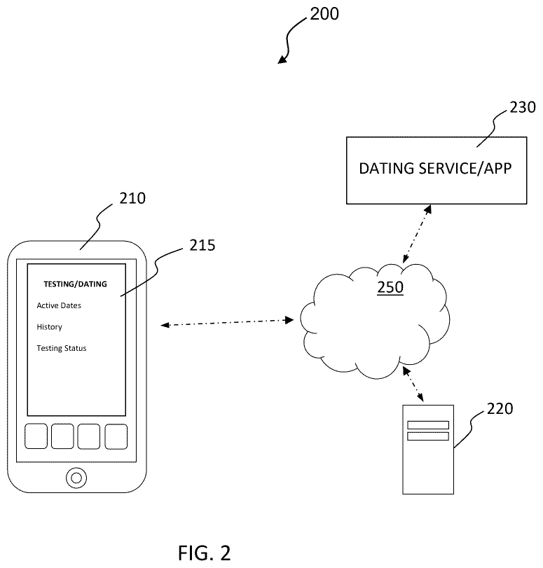

Referring to FIG. 2, illustrated is a diagram 200 for a system in accordance with the embodiments. A user can utilize a mobile device 210 to access an APP 215. The App 215 provides the user with access to dating apps and testing services (together or separately (from which the user can obtain status regarding current dating, dating history, testing status, healthcare support, etc. The user can access testing services 120 and dating services 130 remotely via data network 150. Treatment option after a positive diagnosis can be provided via telehealth and other remote means. User can also have an option of speaking with healthcare providers for immediate treatment.

Note that each the illustrated components or blocks herein may be implemented as modules. Although not required, the disclosed embodiments can be described in the general context of computer-executable instructions, such as program modules, being executed by a single computer or a group of computers. In most instances, such a “module” or component may constitute a software application but can also be implemented as both software and hardware (i.e., a combination of software and hardware).

Generally, program modules include, but are not limited to, routines, subroutines, software applications, programs, objects, components, data structures, etc., that perform particular tasks or implement particular data types and instructions. Moreover, those skilled in the art will appreciate that the disclosed method and system may be practiced with other computer system configurations, such as, for example, hand-held devices, multi-processor systems, data networks, microprocessor-based or programmable consumer electronics, networked PCs, minicomputers, mainframe computers, servers, and the like.

Note that the term module as utilized herein may refer to a collection of routines and data structures that perform a particular task or implements a particular data type. Modules may be composed of two parts: an interface, which lists the constants, data types, variable, and routines that can be accessed by other modules or routines, and an implementation, which may be typically private (accessible only to that module) and which includes source code that actually implements the routines in the module. The term module may also simply refer to an application, such as a computer program designed to assist in the performance of a specific task, such as word processing, accounting, inventory management, etc.

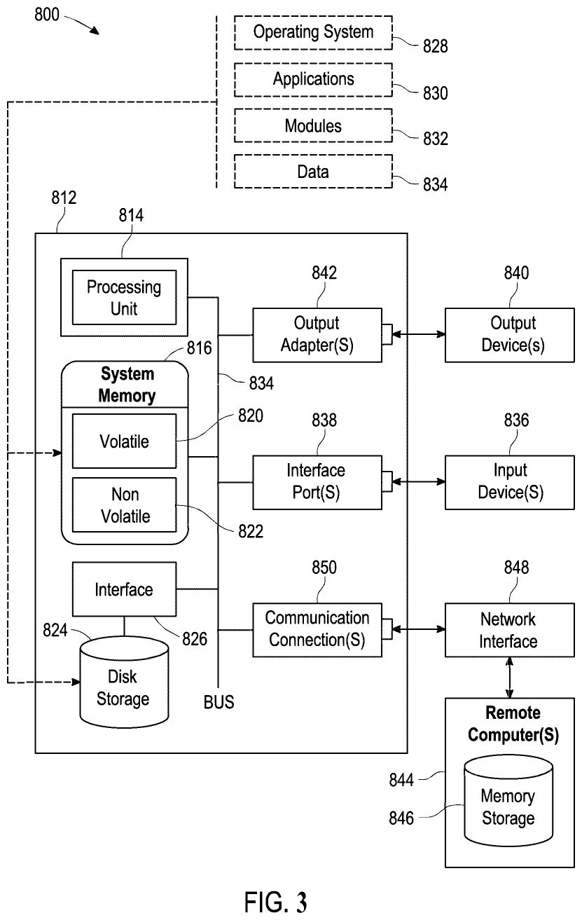

In some example embodiments, the term “module” can also refer to a modular hardware component or a component that is a combination of hardware and software. It should be appreciated that implementation and processing of such modules or components according to the approach described herein can lead to improvements in processing speed and in energy savings and efficiencies in a data-processing system such as, for example, the system or operating environment 800 discussed below with respect to FIG. 3. The various components of the embodiments when integrated as disclosed herein may lead to improvements in, for example, data retrieval speed and a reduction in latency.

In the description herein, specific details of various embodiments are provided. However, some embodiments may be practiced with less than all of these specific details. In other instances, certain methods, procedures, components, structures, and/or functions are described in no more detail than to enable the various embodiments, for the sake of brevity and clarity.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

It should also be noted that at least some of the operations for the methods described herein may be implemented using software instructions stored on a computer useable storage medium for execution by a computer. As an example, an embodiment of a computer program product includes a computer useable storage medium to store a computer readable program.

FIG. 3 illustrates an example of a suitable operating environment 800 for implementing various aspects of this disclosure and which can also include a computer 812. The computer 812 can also include a processing unit 814, a system memory 816, and a system bus 834. The system bus 834 couples system components including, but not limited to, the system memory 816 to the processing unit 814. The processing unit 814 can be any of various available processors. Dual microprocessors and other multiprocessor architectures also can be employed as the processing unit 814.

The system bus 834 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1094), and Small Computer Systems Interface (SCSI). The system memory 816 can also include volatile memory 820 and nonvolatile memory 822. The basic input/output system (BIOS), containing the basic routines to transfer information between elements within the computer 812, such as during start-up, is stored in nonvolatile memory 822.

By way of illustration, and not limitation, nonvolatile memory 822 can include read only memory (ROM), programmable ROM (PROM), electrically programmable ROM (EPROM), electrically erasable programmable ROM (EEPROM), flash memory, or nonvolatile random-access memory (RAM) (e.g., ferroelectric RAM (FeRAM). Volatile memory 820 can also include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 812 can also include removable/non-removable, volatile/non-volatile computer storage media. FIG. 3 illustrates, for example, a disk storage 824. Disk storage 824 can also include, but is not limited to, devices like a magnetic disk drive, floppy disk drive, tape drive, Jaz drive, Zip drive, LS-100 drive, flash memory card, or memory stick. The disk storage 824 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 824 to the system bus 834, a removable or non-removable interface is typically used, such as interface 826.

FIG. 3 also depicts software that can act as an intermediary between users and the basic computer resources described in the suitable operating environment 800. Such software can also include, for example, an operating system 828. Operating system 828, which can be stored on disk storage 824, acts to control and allocate resources of the computer 812. System applications 830 can take advantage of the management of resources by operating system 828 through program modules 832 and program data 834, e.g., stored either in system memory 816 or on disk storage 824. It is to be appreciated that this disclosure can be implemented with various operating systems or combinations of operating systems.

A user can enter commands or information into the computer 812 through input device(s) 836. Input device(s) 836 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices can connect to the processing unit 814 through the system bus 834 via interface port(s) 838. Interface port(s) 838 can include, for example, a serial port, a parallel port, a game port, and a universal serial bus (USB). Output device(s) 840 may use some of the same type of ports as input device(s) 836.

Thus, for example, a USB port can be used to provide input to computer 812, and to output information from computer 812 to an output device 840. Output adapter 842 is provided herein to illustrate that there may be some output devices 840 like monitors, speakers, and printers, among other output devices 840, which require special adapters. The output adapters 842 include, by way of illustration and not limitation, video and sound cards that provide a means of connection between the output device 840 and the system bus 834. It should be noted that other devices and/or systems of devices provide both input and output capabilities such as remote computer(s) 844.

Computer 812 can operate in a networked environment using logical connections to one or more remote computers, such as remote computer(s) 844. The remote computer(s) 844 can be a computer, a server, a router, a network PC, a workstation, a microprocessor-based appliance, a peer device or other common network node and the like and typically can also include many or all the elements described relative to computer 812. For purposes of brevity, only a memory storage device 846 is illustrated with remote computer(s) 844. Remote computer(s) 844 can be logically connected to computer 812 through a network interface 848 and then can be physically connected via communication connection 850.

Network interface 848 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 850 refers to the hardware/software employed to connect the network interface 848 to, for example, the system bus 834. While communication connection 850 is shown for illustrative clarity inside computer 812, it can also be external to the computer 812. The hardware/software for connection to the network interface 848 can also include, for exemplary purposes only, internal and external technologies such as, modems including regular telephone grade modems, cable modems and DSL modems, ISDN adapters, and Ethernet cards.

Embodiments may be implemented in or as a system, a method, an apparatus and/or a computer program product at any possible technical detail level of integration. The computer program product can include a computer readable storage medium (or media) having computer readable program instructions thereon for causing a processor to carry out aspects of the embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in one or more computing/processing device receives computer readable program instructions from the network and forwards the computer readable program instructions for storage in a computer readable storage medium within the respective computing/processing device.

Computer readable program instructions for carrying out operations of various aspects of the embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages.

The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to customize the electronic circuitry, to perform aspects of the embodiments.

Aspects of the embodiments are described herein with reference to flowchart illustrations and/or block diagrams of methods, apparatus (systems), and computer program products according to embodiments of the invention. It can be understood that one or more blocks of the flowchart illustrations and/or block diagrams, and combinations of blocks in the flowchart illustrations and/or block diagrams, can be implemented by computer readable program instructions. These computer readable program instructions can be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions can also be stored in a computer readable storage medium that can direct a computer, a programmable data processing apparatus, and/or other devices to function in a manner, such that the computer readable storage medium having instructions stored therein comprises an article of manufacture including instructions which implement aspects of the function/act specified in the flowchart and/or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the embodiments. In this regard, one or more blocks in the flowchart or block diagrams can represent a module, segment, or portion of instructions, which comprises one or more executable instructions for implementing the specified logical function(s).

In some alternative implementations of the embodiments, the functions noted in the blocks can occur out of the order noted in the figures. For example, two blocks shown in succession can, in fact, be executed substantially concurrently, or the blocks can sometimes be executed in the reverse order, depending upon the functionality involved. It can also be noted that one or more block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer and/or computers, those skilled in the art can recognize that this disclosure also can or can be implemented in combination with other program modules. Generally, program modules include routines, programs, components, data structures, etc. that perform particular tasks and/or implement abstract data types. Moreover, those skilled in the art can appreciate that the inventive computer-implemented methods can be practiced with other computer system configurations, including single-processor or multiprocessor computer systems, mini-computing devices, mainframe computers, as well as computers, hand-held computing devices (e.g., PDA, cellular phone, etc.), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” “module,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer.

By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal).

As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a server computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, the term “processor” can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein.

Further, processors can exploit nano-scale architectures such as, but not limited to, molecular and quantum-dot based transistors, switches and gates, to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random-access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

Based on the foregoing, it can be appreciated that a number of embodiments including preferred and alternative embodiments, are disclosed herein. For example, in an embodiment, a method for providing PCT (Preventive Care and Testing) services to customers, can involve: receiving user data from a plurality of users, including personal information and medical history; conducting PCT testing on the users for Sexually Transmitted Diseases (STDs); generating a certification indicating the STD-free status of the users based on the results of the PCT testing; coordinating with dating services to incorporate the certification into user profiles, thereby indicating to potential partners the STD-free status of the users.

In an embodiment, a system for coordinating PCT testing with dating services to certify the STD-free status of users, can include: a database for storing user data, including personal information, medical history, and PCT test results; a PCT testing module configured to conduct STD testing on the users and generate test results; a certification generation module configured to create certifications indicating the STD-free status of the users based on the test results; and an interface module configured to communicate with dating services to integrate the certifications into user profiles.

In an embodiment, a computer-readable storage medium can be implemented, which can store instructions that, when executed by a processor, cause the processor to perform a method for certifying the STD-free status of users on dating services, the method comprising: receiving user data from a plurality of users, including personal information and medical history; conducting PCT testing on the users for STDs; analyzing the results of the PCT testing to determine the STD-free status of the users; generating a certification indicating the STD-free status of the users; and transmitting the certification to dating services for incorporation into user profiles.

The embodiments therefore relate to a method, system, and computer-readable medium for providing Preventive Care and Testing (PCT) services to customers, particularly for testing and certifying the sexually transmitted disease (STD)-free status of users. The disclosed technology facilitates the secure collection, processing, and integration of user health data with dating services, thereby enabling individuals to display verified STD-free certifications on their dating profiles. The embodiments aim to promote transparency and health awareness while ensuring user privacy and data security.

In one embodiment, the disclosed technology provides a method for delivering PCT services to users. The method includes receiving user data from a plurality of users, wherein the data comprises personal information and medical history. The system then facilitates PCT testing for STDs through certified laboratories or medical facilities. Upon obtaining the test results, a certification is generated to indicate the STD-free status of the user. The certification is then integrated with dating services, allowing users to display verified health statuses within their profiles. The process ensures that users seeking potential partners on dating platforms have access to verified health information, enhancing confidence and safety in online dating interactions.

Another embodiment provides a system configured to coordinate PCT testing with dating services for certifying users' STD-free status. The system comprises a database for securely storing user data, including personal details, medical history, and test results. A PCT testing module facilitates the conduction of STD testing for users through authorized medical providers. Upon processing the test results, a certification generation module generates a digital certification that confirms the STD-free status of the tested individual. An interface module enables communication with dating services, allowing seamless integration of the certification into user profiles. The system employs encryption and access control mechanisms to protect user privacy and ensure that only authorized entities can access sensitive medical information.

In yet another embodiment, a computer-readable storage medium stores instructions that, when executed by a processor, enable a system to perform the method for certifying the STD-free status of users on dating services. The process involves receiving user data from multiple users, including personal and medical information, and conducting PCT testing for STDs. The results are then analyzed to determine whether a user meets the STD-free criteria. Upon validation, a certification is generated and transmitted to dating platforms for integration into user profiles. The software ensures the secure transmission of medical data while maintaining compliance with applicable healthcare data protection regulations.

To ensure the confidentiality and authenticity of user certifications, the system may incorporate encryption techniques, blockchain-based verification, or biometric authentication for secure user access. Additionally, users may be provided with control options to manage who can view their certifications on dating platforms. The system may also support periodic re-testing reminders and automated updates to maintain the relevance of displayed certifications.

By integrating health certification services with dating platforms, the disclosed technology enhances transparency and promotes safer dating environments while addressing privacy concerns through advanced security measures.

The disclosed embodiments provide several advantages, particularly in promoting transparency and public health awareness in online dating. By integrating verified STD-free certifications into user profiles, the system fosters trust among individuals seeking potential partners while encouraging regular preventive testing. The process is designed to be seamless and automated, allowing users to undergo testing, receive certifications, and integrate them with dating platforms without manual intervention. Additionally, the system enhances data security and confidentiality through encryption, controlled access, and potential blockchain-based verification, ensuring that sensitive medical information is protected while enabling controlled sharing.

Beyond dating applications, the embodiments have the potential to expand into other areas such as workplace health verifications, travel requirements, and community health programs. Users can benefit from built-in reminders for periodic re-testing, helping them maintain up-to-date certifications and remain informed about their health status. By working with certified laboratories and medical providers, the system can ensure the accuracy and reliability of the issued certifications while minimizing misinformation or fraudulent health claims. Customizable privacy controls empower users to manage who can access their health information, balancing transparency with personal privacy.

The PCT testing module is a core component of the disclosed system, responsible for conducting STD testing and ensuring accurate, reliable results before certification. This module can be configured to interface with certified laboratories, medical providers, and user devices to facilitate the collection, processing, and verification of test results in a secure and efficient manner.

The module operates in multiple stages, beginning with user registration and test initiation. Users provide personal information and medical history, which the system securely stores and associates with their test requests. The module then interfaces with partnered healthcare providers or testing facilities, generating test orders and directing users to approved locations for sample collection, which may include blood, urine, or swab tests depending on the type of STD screening required.

Once samples are collected, the testing module processes and analyzes the results using certified laboratory protocols. The results undergo automated verification to confirm accuracy and ensure compliance with medical standards. The module may incorporate artificial intelligence (AI) or machine learning techniques to detect anomalies, flag potential retests, or assess trends in user health data for proactive recommendations.

Following result validation, the certification generation process is initiated. If a user meets the STD-free criteria, the module creates a digital certification confirming their status. This certification can be securely stored and can be accessed through the system's interface module for controlled sharing with dating services. The module can also include features for secure result delivery, allowing users to view their test outcomes through encrypted channels while maintaining strict privacy controls.

Additionally, the PCT testing module can support periodic retesting in some embodiments by sending reminders to users when their certification is about to expire, ensuring ongoing preventive care. The system may also integrate with electronic health record (EHR) systems to facilitate seamless medical history updates and may offer an optional feature for telemedicine consultations if users require further medical guidance.

Overall, the PCT testing module can ensure a secure, automated, and user-friendly approach to STD testing, reducing barriers to preventive healthcare while enhancing transparency in online dating and other social interactions.

Based on the foregoing, it can be appreciated that a number of embodiments are disclosed herein including preferred and alternative embodiments. For example, in an embodiment, a method for providing PCT (Preventive Care and Testing) services to customers, can involve: receiving user data from a plurality of users, including personal information and medical history; conducting PCT testing on the users for Sexually Transmitted Diseases (STDs); generating a certification indicating the STD-free status of the users based on the results of the PCT testing; and coordinating with dating services to incorporate the certification into user profiles, thereby indicating to potential partners the STD-free status of the users.

An embodiment can involve encrypting and securely storing user data, including test results, to ensure privacy and compliance with data protection regulations.

An embodiment can also involve integrating the certification into a user's dating profile as a visually distinguishable verification badge indicating their STD-free status.

An embodiment may further involve providing users with an option to share their certification status with specific individuals through a secure, permission-based system.

In an embodiment, coordinating with dating services can further involve using an application programming interface (API) to automatically update user profiles with certification status.

In an embodiment, a system for coordinating PCT testing with dating services to certify the STD-free status of users, can include: a database for storing user data, including personal information, medical history, and PCT test results; a PCT testing module configured to conduct STD testing on the users and generate test results; a certification generation module configured to create certifications indicating the STD-free status of the users based on the test results; and an interface module configured to communicate with dating services to integrate the certifications into user profiles.

In an embodiment, a computer-readable storage medium can be implemented, which stores instructions that, when executed by a processor, cause the processor to perform a method for certifying the STD-free status of users on dating services, the method comprising: receiving user data from a plurality of users, including personal information and medical history; conducting PCT testing on the users for STDs; analyzing the results of the PCT testing to determine the STD-free status of the users; generating a certification indicating the STD-free status of the users; and transmitting the certification to dating services for incorporation into user profiles.

It will be appreciated that variations of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the following claims.

What is claimed is:

1. A method for providing PCT (Preventive Care and Testing) services to customers comprising:
receiving user data from a plurality of users, including personal information and medical history;
conducting PCT testing on the users for Sexually Transmitted Diseases (STDs);
generating a certification indicating the STD-free status of the users based on the results of the PCT testing;
coordinating with dating services to incorporate the certification into user profiles, thereby indicating to potential partners the STD-free status of the users.

2. The method of claim 1, further comprising encrypting and securely storing user data, including test results, to ensure privacy and compliance with data protection regulations.

3. The method of claim 1, further comprising integrating the certification into a user's dating profile as a visually distinguishable verification badge indicating their STD-free status.

4. The method of claim 1, further comprising providing users with an option to share their certification status with specific individuals through a secure, permission-based system.

5. The method of claim 1, wherein coordinating with dating services further comprises using an application programming interface (API) to automatically update user profiles with certification status.

6. A system for coordinating PCT testing with dating services to certify the STD-free status of users, comprising:
a database for storing user data, including personal information, medical history, and PCT test results;
a PCT testing module configured to conduct STD testing on the users and generate test results;
a certification generation module configured to create certifications indicating the STD-free status of the users based on the test results;
an interface module configured to communicate with dating services to integrate the certifications into user profiles.

7. A computer-readable storage medium storing instructions that, when executed by a processor, cause the processor to perform a method for certifying the STD-free status of users on dating services, the method comprising:
receiving user data from a plurality of users, including personal information and medical history;
conducting PCT testing on the users for STDs;
analyzing the results of the PCT testing to determine the STD-free status of the users;
generating a certification indicating the STD-free status of the users;
transmitting the certification to dating services for incorporation into user profiles.
